(19) United States
(12) Patent Application Publication
(10) Pub. No.: US 2022/0338722 A1
(43) Pub. Date: Oct. 27, 2022

(54) MICROPLASMA INTEGRATED ARRAY OTOSCOPE SPECULUM AND EAR TREATMENT METHODS

(71) Applicant: The Board of Trustees of the University of lllinois, Urbana, IL (US)

(72) Inventors: J. Gary Eden, Urbana, IL (US); Peter P. Sun, Urbana, IL (US); Wenyuan Chen, Urbana, IL (US); Thanh H. Nguyen, Urbana, IL (US); Guillermo L. Monroy, Urbana, IL (US); Jungeun Won, Urbana, IL (US); Stephen A. Boppart, Urbana, IL (US)

(21) Appl. No.: 17/621,803

(22) PCT Filed: Jun. 24, 2020
(86) PCT No.: PCT/US2020/039250

§ 371 Date: Dec. 22, 2021

Related U.S. Application Data
(60) Provisional application No. 62/866,707, filed on Jun. 26, 2019.

(51) Int. Cl. A61B 1/227; A61N 1/44; H05H 1/24

(52) U.S. Cl. CPC A61B 1/227; A61N 1/44; H05H 1/2431; H05H 2245/30

(57) ABSTRACT

A speculum body is configured to attach to the otoscope. An array of radially situated microchannels is within the speculum body and extends to apertures in a distal end of the speculum body. A power electrode array is within the speculum body positioned with respect to the microchannels to excite plasma generation within the microchannels. An optically transparent central portion is in the body to permit viewing of an eardrum by a practitioner. A method of treatment of the middle ear and/or middle ear cavity incudes actuating plasma jets to extend into the ear canal from a speculum attached to the otoscope and continuing the plasma jet treatment for a period of time sufficient to inactivate or kill a bacterial biofilm in the middle ear and/or the middle ear cavity.

### PRIORITY CLAIM AND REFERENCE TO RELATED APPLICATION

The application claims priority under 35 U.S.C. § 119 and all applicable statutes and treaties from prior U.S. provisional application Ser. No. 62/866,707, which was filed Jun. 26, 2019.

### STATEMENT OF GOVERNMENT INTEREST

This invention was made with government support under grant number FA9550-14-1-0002 awarded by the U.S. Air Force Office of Scientific Research, and under 1 ROl EB013723 awarded by the National Institutes for Health. The government has certain rights in the invention.

### FIELD

Fields of the invention include otoscopes, ear treatment methods, and microplasma arrays.

### BACKGROUND

Middle ear infections affect more than 80% of children in the United States, and the common treatment for an acute middle ear infection is to prescribe antibiotics. Antibiotic treatment has been shown to be ineffective in over 30% of the cases involving acute middle ear infections. Chronic ear infections have been associated with the development of a bacterial biofilm in the middle ear space, and the bacteria within these biofilms develop antibiotic resistance which leads to recurrent ear infections. Myringotomy followed by the placement of a tympanostomy tube is a surgical method of treatment that creates an incision on the ear drum for drainage, and is the common procedure to arrest chronic ear infections.

### SUMMARY OF THE INVENTION

A preferred embodiment provides a speculum body that is configured to attach to the otoscope. An array of radially situated microchannels is within the speculum body and extends to apertures in a distal end of the speculum body. A power electrode array is within the speculum body positioned with respect to the microchannels to excite plasma generation within the microchannels. An optically transparent central portion is in the body to permit viewing of an eardrum by a practitioner. A method of treatment of the middle ear and/or middle ear cavity includes actuating plasma jets to extend into the ear canal from a speculum attached to the otoscope. Plasma jet treatment of the eardrum for a sufficient period of time inactivates or kills a bacterial biofilm in the middle ear and/or the middle ear cavity.

### BRIEF DESCRIPTION OF THE DRAWINGS

FIGS. 1A-1H illustrate a preferred microplasma-jet-array integrated speculum and otoscope system for probing and treating middle ear infections;

FIG. 2A is a partial transparent perspective view of an electrode array section of a preferred microplasma-jet-array integrated speculum; FIG. 2B is a perspective view of a preferred shape for a microplasma-jet-array integrated speculum suited for pneumatic otoscopy;

FIGS. 3A-3F are views of a gas feed section of a preferred microplasma jet array-integrated speculum;

FIGS. 4A-4G are views of an otoscope adapter section of a preferred microplasma jet array-integrated speculum;

FIG. 5 is a plot of the colony diameter of P. aeruginosa before and after microplasma jet array treatment;

FIGS. 6A and 6B present data of the drug susceptibility of P. aeruginosa planktonic cells (FIG. 6A), and the biofilm (FIG. 6B) four days after microplasma jet array treatments.

### DETAILED DESCRIPTION OF THE PREFERRED EMBODIMENTS

A preferred embodiment is an otoscope having a microplasma-integrated speculum. The speculum includes microchannels dispersed so as to approximate a cone, and plasma is generated in each microchannel. A power electrode array is positioned in a similar manner between the microchannels. The speculum can be formed from a plurality of sections or can be formed as a single unitary structure. The speculum has a hollow center to permit a practitioner to view the eardrum optically. A switch can be provided to activate microplasma generation by controlling power to the electrodes and plasma medium flow in the microchannels. The microchannels together form an array of jets of plasma ejected from the microchannels by gas flowing through the microchannels. This low temperature plasma is directed by the otoscope to the vicinity of the outer surface of the eardrum. Molecular radicals produced by the interaction of the plasmas with room air are able to diffuse through the eardrum and deactivate a biofilm located on the inner surface of the eardrum in the middle ear. The speculum can, in preferred embodiments, be disposable and can be fabricated via 3D printing.

In preferred embodiments, the microplasma-integrated speculum includes an outer rubber membrane shaped and configured to create a seal within the ear canal, which is commonly done by pneumatic otoscopy in which induced pressure changes serve to visualize any displacement of the ear drum. The sealed ear canal in a preferred method for biofilm treatment in the middle ear facilitates the accumulation of gaseous, reactive nitrogen or oxygen-bearing radicals generated by the plasma, thereby allowing them to diffuse through the ear drum and into the middle ear space. Preferred methods can kill or inactivate bacteria in the middle ear and middle ear cavity. Those bacteria, if residing sufficiently long in the middle ear, may also form a biofilm. Ear infections are caused by many types of bacteria, of which Pseudomonas is a primary example. The molecular species produced from room air or other gas mixtures by plasma jets can, however, kill or inactivate many types of bacteria. Ear infections can also be caused by viruses, and plasma is reported in the literature to effectively kill or inactivate viruses. Preferred treatments can be adjusted by the time duration as well as the voltage or rate of generation of the plasma species. Preferred methods include multiple treatments over time.

The speculum preferably includes portable and replaceable microplasma jet arrays that can be fabricated by 3D printing. Extensive testing has demonstrated the efficacy of the otoscope plasma-generated molecular species in treating middle ear infections. The antibiotic susceptibility of Pseudomonas aeruginosa, a common bacterial strain associated with middle ear infections, was measured with the minimal inhibitory concentration (MIC₅₀) that causes 50% growth inhibition and was found to fall by a factor of five after 10 minutes of plasma treatment. After 12 minutes of plasma treatment, the MIC₅₀ dropped by more than three orders-of-magnitude. The number of living cells remaining in the cultured bacterial biofilm before and after microplasma jet array treatment has been investigated through confocal laser scanning microscopy. Reactive species, such as OH and ¹O₂ produced by the microplasma jet array and evaluated quantitatively through liquid chromatography, are believed to play an important role during disinfection. Testing has consistently revealed that an otoscope of the invention can provide an effective treatment method for middle ear infections.

Preferred embodiments of the invention will now be discussed with respect to the drawings and experiments used to demonstrate the invention. The drawings may include schematic representations, which will be understood by artisans in view of the general knowledge in the art and the description that follows.

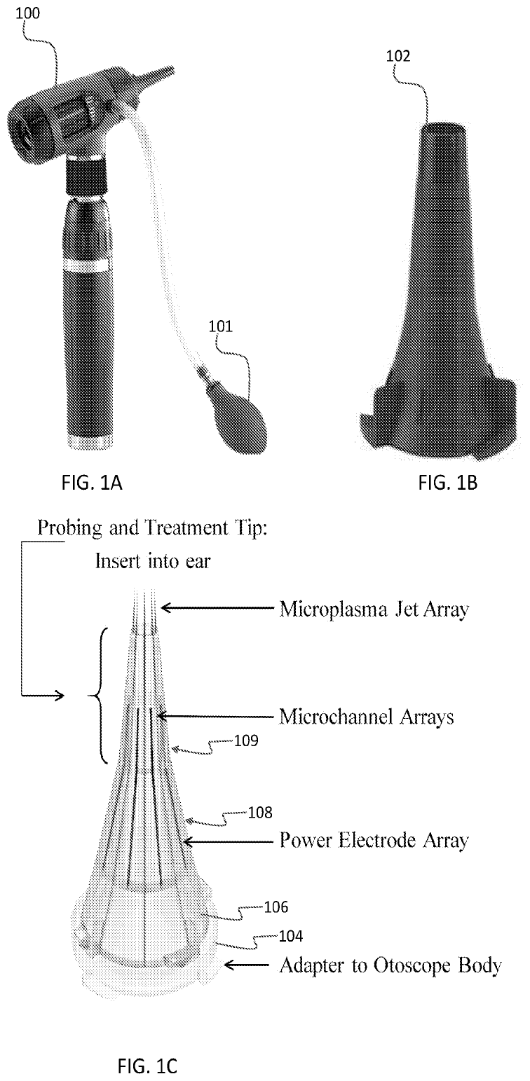

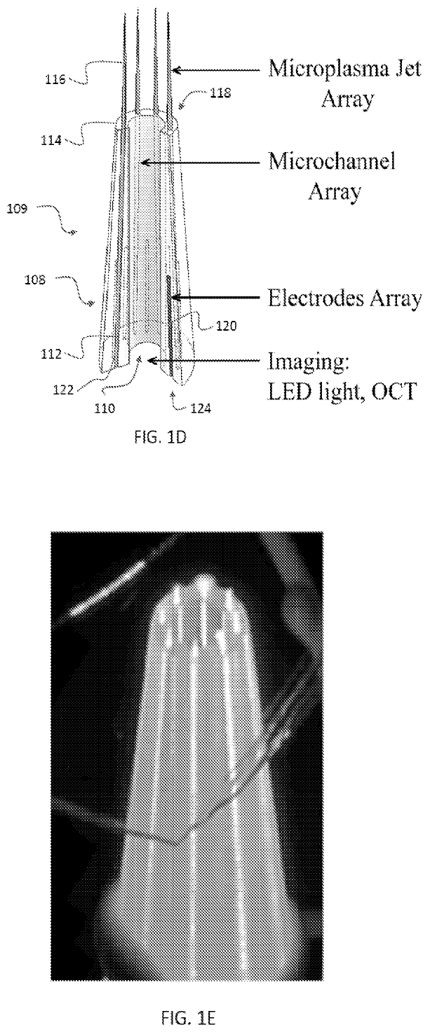

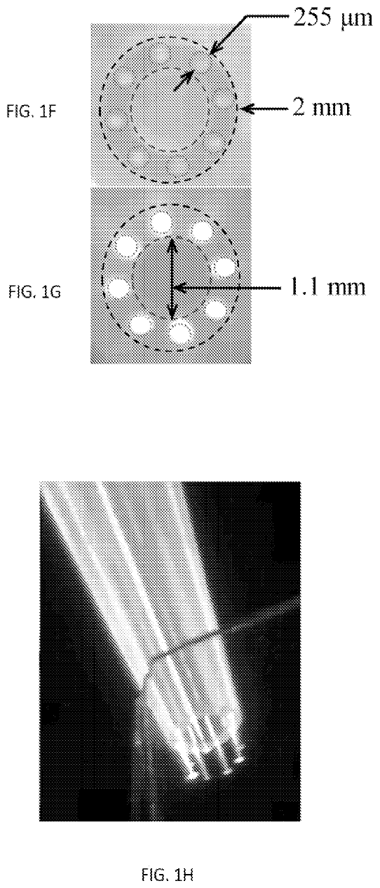

FIGS. 1A-1H illustrate a preferred microplasma jet array-integrated speculum and otoscope system for probing and treating middle ear infections. A conventional otoscope 100, shown in FIG. 1A, is a common component of a physician's toolkit and provides illumination and optical magnification so as to allow a detailed view of the outer surface of the eardrum. The otoscope 100 includes an inflation bulb 101, which can be used to perform pneumatic otoscopy. The inner surface of the eardrum is part of the middle ear cavity or space and is not optically accessible. To prevent contamination of the otoscope, and infectious agents transmitted between subjects, a typical otoscope speculum is disposable, and can be easily removed from the otoscope by a rotational snap-fit mechanism. Preferred embodiments provide a significant design change to the speculum by incorporating microplasma jet arrays for treatment of middle ear infections while maintaining the original purpose of viewing the eardrum and/or performing pneumatic otoscopy. The present specula can be manufactured by three-dimensional (3D) printing techniques and are also disposable. FIG. 1B is a perspective view of a preferred unitary speculum 102, and FIG. 1C shows portions and details of the speculum 102, including the body of speculum 102 that includes a separate adapter 104, gas feed section 106, and power section 108 with tip 109. In the prototype, the power section 108 was integrated with the tip 109, as shown in FIG. 1D, but they may also be separate and designed to be connected. The adapter 104 and gas feed section 106 were also distinct components of the speculum but could also be formed in a unitary structure with the tip 109. The tip 109 is shown in a partial cut-away view in FIG. 1D. FIG. 1E shows the tip of the experimental disposable, 3D-printed speculum prototype device in operation with plasma emanating from the speculum. For this particular test and illustrative purposes, neon gas was flowed through the microchannels and provided the medium in which plasma was generated. However, many gases and gas mixtures may be used (such as the rare gases, nitrogen, and air) but air is most economical and allows for one to dispense with an external gas cylinder. Furthermore, plasmas generated in room air produce the OH and NO radicals that are believed to be most effective in deactivating the pathogens causing ear infections. For the sake of clarity, the gas feed tubing and electrical wiring diagrams are not shown in the figure. These components can be located within the otoscope or exterior to it in such a way as to not obscure the optical path. A mass flow meter can be used to control the gas input into microchannels, as demonstrated in experiments where both pressure and flow ratio were monitored. Additional plasma media tested included He, Ar, and mixtures of O₂ with these rare gases, or simply nitrogen mixed with oxygen. Air can also be a carrier gas together with other gases (such as water vapor from which the OH radical is produced by a low temperature plasma) or by itself. A plasma medium can be selected to target particular types of ear-infections, i.e., what type of bacteria are involved. Different plasma media will produce different plasma chemistries, and the efficacy of different chemistries can be tested with common types of ear-infection bacteria to determine which provides the most effective plasma chemistry for treatment.

With reference to FIG. 1D, the tip 109 is in the general form of a cone with a lumen 110 situated along the axis of the cone to permit the normal imaging function of the otoscope 100. The lumen 110 provides an optical path for viewing a patient's ear drum, so the lumen 110 could also be an optically transparent material or one or more lenses. The tip 108 includes a plurality of radially disposed microchannels 112 that extend longitudinally the full length of the tip 108, and includes apertures 114 at its distal end for ejecting microplasma in the form of jets 116 away from a distal end 118 of the tip 108. Interspersed between the radially-arranged array of microchannels 112 is an array of radially-disposed electrodes 120, physically isolated from the microchannels by the material from which the tip 108 is fabricated. Inlets 122 at a proximal end permit a plasma medium—a gas or a mixture of gases and/or vapors- to flow into the array of microchannels 112 in which an appropriate time-varying voltage applied to the array of electrodes 120 generates the microplasma jets 116.

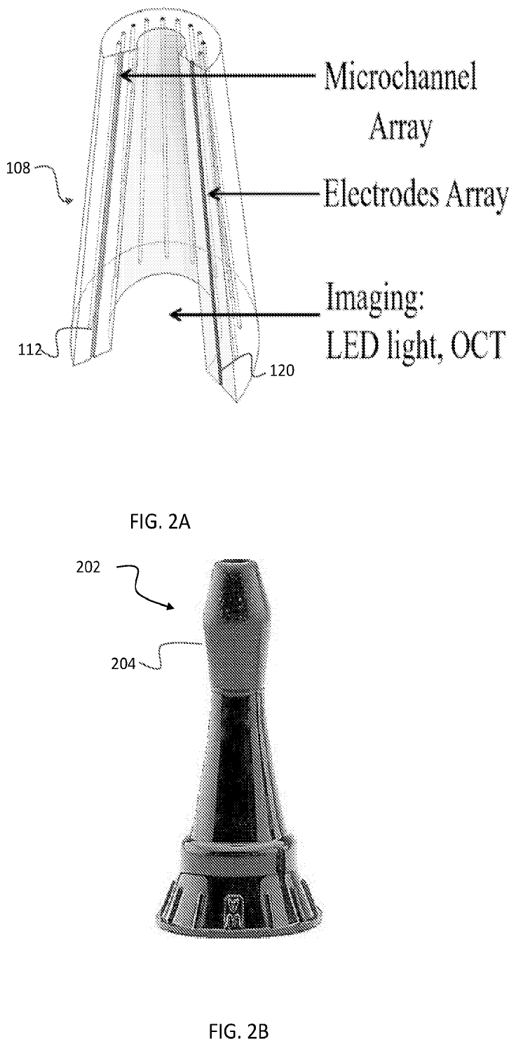

FIGS. 1F and 1G provide several dimensions of the prototype speculum tip, and FIG. 1H provides another view of this embodiment in operation. The prototype speculum tip consisted of conically situated microchannels. In the prototype, the diameter of each of 8 separate microchannels was 255 μm. As shown in FIGS. 1F and 1G, an inner diameter and outer diameter of the tip (distal end of tip 108) are 1.1 mm and 2.5 mm, respectively, and the aperture of the microchannels is 255 μm. As also shown in FIG. 2A, the power electrode array was also radially disposed such that an electrode 120 was disposed between each of the microchannels 112. The electrodes need only extend the length of the electrode array section 108, and it is not necessary for them to extend all the way to the distal end 118. Generally, the ratio/length of the electrodes compared to the microchannels is dependent on the plasma medium, its flow, microchannel cross-section and power level, as known in the art of microplasma jets. See, e.g. Eden et al. U.S. Pat. No. 8,957,572, entitled Microplasma jet devices, arrays, medical devices and methods; P. P. Sun, J. H. Cho, C. H. Park, S. J. Park, and J. G. Eden. “Close-packed arrays of plasma jets emanating from microchannels in a transparent polymer.” IEEE Transactions on Plasma Science 40, no. 11 (2012): 2946-2950. Preferably, to ensure electrical safety, sufficient electrode-free length is provided (e.g., 1.5-2.5 cm) so as to avoid having the electrode array section 108 inserted into ear.

FIG. 2B shows a preferred outer shape for a preferred speculum 202 of the invention. The inner structure of the speculum 202 includes the features of FIGS. 1C-2A for generating the microplasma jets, while FIG. 2B is a perspective view to illustrate an outer profile of the speculum 202, which is configured and shaped so as to create an airtight seal made by an ear tip 204 in the ear canal. During an examination and treatment, a doctor hand-squeezes the bulb 101 (FIG. 1A) to change the pressure in the ear canal while observing whether the ear drum moves with changing pressure. Lack of movement of the ear drum indicates the presence of fluid and pressure in the middle ear which is a pathological condition. For the purposes of a preferred method of biofilm treatment in the inner ear, sealing of the ear canal with speculum 202 is advantageous in allowing the plasma-generated, nitrogen or oxygen-bearing radicals to build up in the canal and subsequently diffuse through the ear drum and into the middle ear where the bacteria/biofilm is located, and also in the middle ear cavity. The size of the speculums 102 and 202 can be selected to match ear canal anatomy, as with conventional speculum sizing. Thus, for example, the specula of the invention can be provided in different sizes/diameters at the tip, to accommodate children-to-adults, or specific anatomical differences, e.g. large canals, small canals, etc.

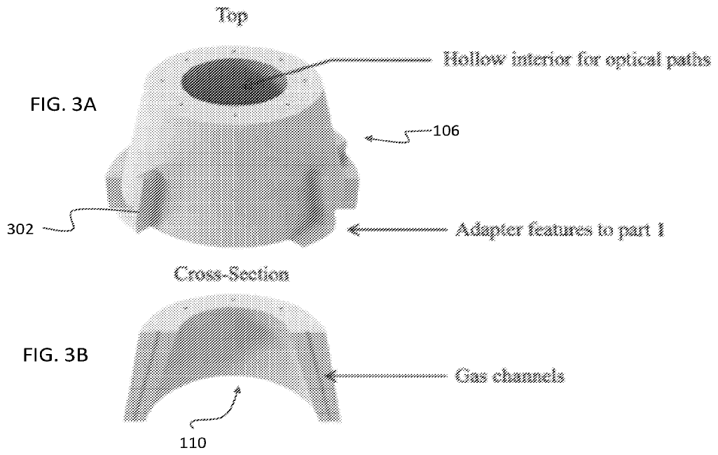

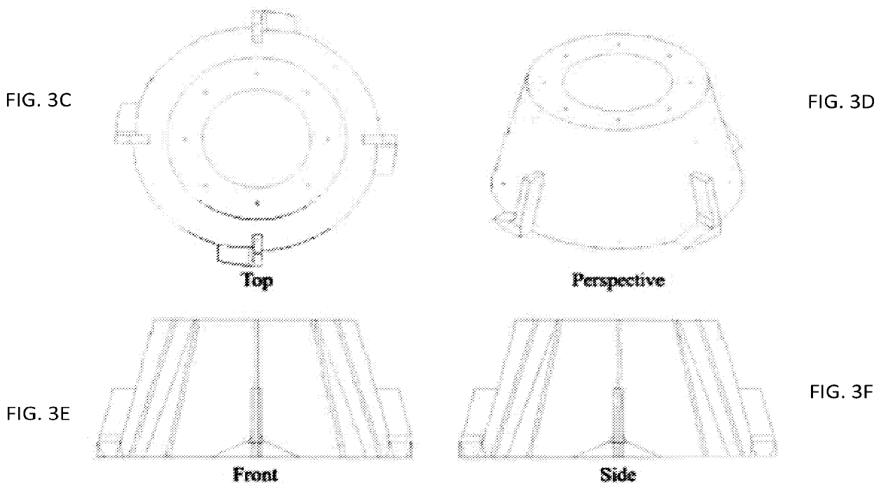

FIGS. 3A-3F show the preferred gas feed section 106, which has a tapered, truncated cone shape. Microchannels 112 extend through the gas feed section to match and mate with those in the electrode array section and permit plasma medium (typically a gas or mixture of gases) to flow from the gas feed section 106 into the electrode array section, and eventually into the tip 109. The lumen 110 also extends through the gas feed section, thus providing a clear optical path from the otoscope 100 to the distal end 118 of the tip 109. The gas feed section 106 includes rotational and positive-lock structures 302 to mate with complementary structures on the adapter 104.

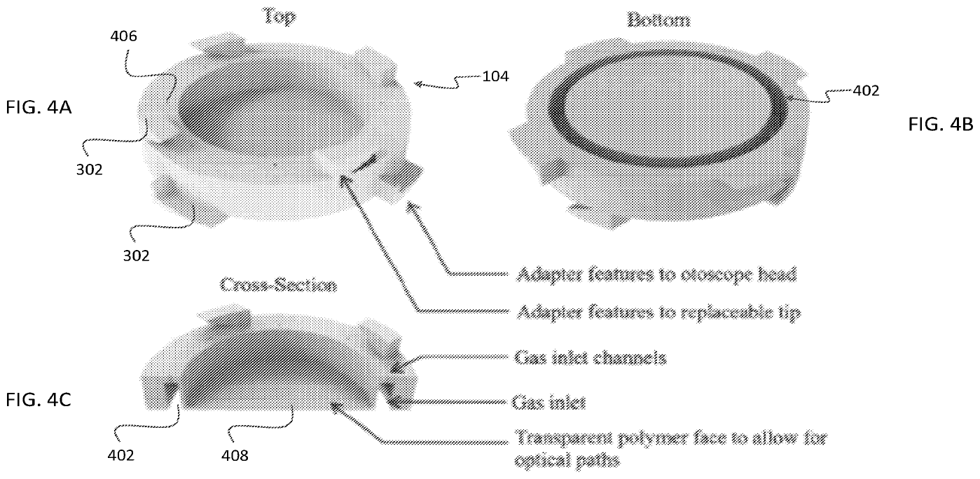

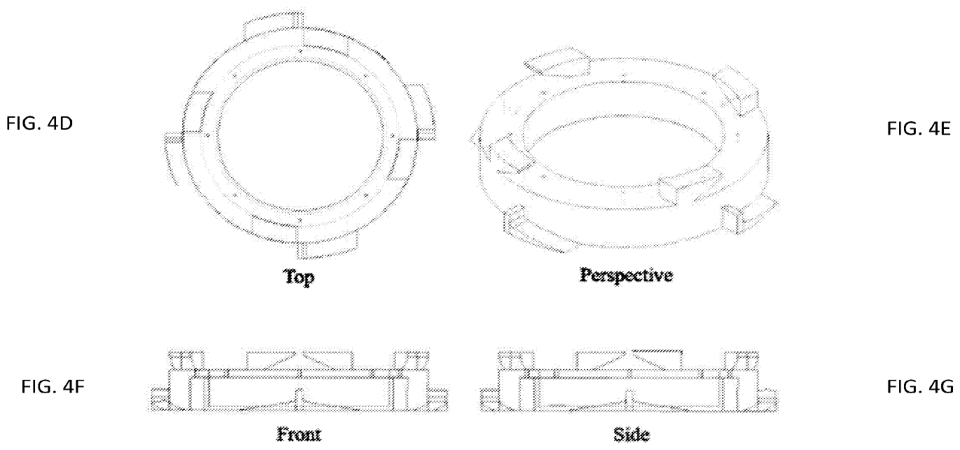

FIGS. 4A-4G show the preferred adapter 104. The adapter 104 includes complementary rotational and positive lock structures 302 on both sides, one side to mate with the gas feed section 106 and the side for the otoscope 100. A bottom side includes a gas inlet circumferential channel 402. Outlets 406 are in fluid communication with the channel 402 and provide plasma medium flow into the microchannels 112 of the gas feed section 106. A center section 408 is optically transparent. The turn (rotational) and positive lock structures 302 permit the adapter 104, gas feed section 106 and a center section 408 to connect to each other. Plasma medium enters the gas inlet circumferential channel 402, flows up through the adapter into the array of microchannels 112, where it is ignited via applied electrical potential in the electrode array section 108. Once plasma is generated in the microchannels, the plasma continues to move at a uniform speed and subsequently emerges from the apertures 114 at the distal end 118 of the tip 109. Plasmas exiting the apertures are often referred to as “plasma jets”.

In Vitro P. aeruginosa Inactivation

The inactivation of P. aeruginosa by microplasma jet array was studied by analyzing different microplasma jet array treatment times. A reduction of 0.6±0.3 logs in the P. aeruginosa bacterial cell count was achieved after 3 minutes of microplasma jet array treatment for a driving voltage to the array of 1.55 kV (RMS) which corresponds to a power density delivered to the array of 0.15 W/cm². A linear reduction trend was observed during the first 3 mins of irradiation time, but no significant reductions were observed from 3 mins to 7 mins of microplasma jet array treatment. Approximately 2.3±0.6 logs of reduction in the P. aeruginosa density was obtained with 10 mins of treatment.

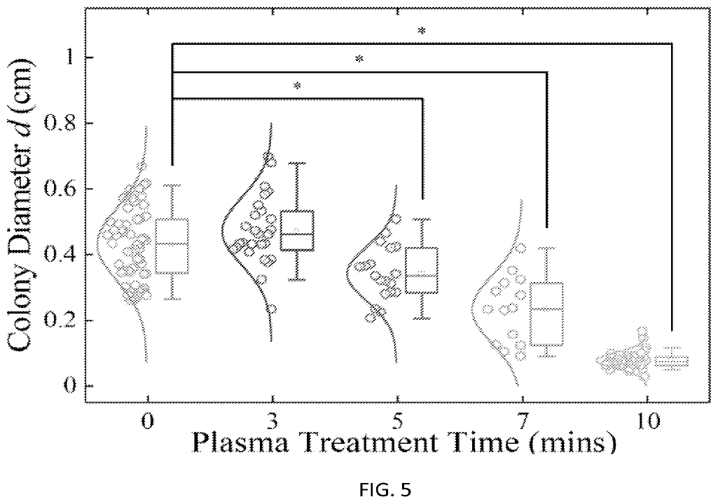

Interestingly, the size of the colony-forming unit, which indicates the growth rates for the cells, changed dramatically after the microplasma jet array treatment. The colony diameters, determined from analysis of the sizes of the colony forming units, are illustrated in FIG. 5. After 16 hours incubation at 37 C, the colony diameter had increased slightly from 0.43±0.11 cm to 0.47±0.1 cm after 3 mins of microplasma jet array treatment, but no other significant differences were observed (p=1.18×10⁻¹). With a further extension of the treatment time from 5 mins to 7 mins to 10 mins (with the same array power density), the colony diameter had decreased from 0.34±0.08 (p=1.8×10⁻³) to 0.23±0.01 (p<<0.05), to 0.08±0.02, as shown in FIG. 5.

Within the first 3 mins, although 0.6 logs reduction was achieved, the colony size remained the same. Extending the treatment time from 5 to 7 mins resulted in an almost imperceptible log reduction (0.48±0.08 to 0.41), but the colony size decreased significantly (p=0.002). However, the colony reduction and colony size both declined after a microplasma jet array treatment of 7 mins and 10 mins to p<<0.05 for each of the 7-minute and 10-minute treatments.

In order to validate the present plasma treatment method, cellular nitrite membranes were used as surrogates to mimic the eardrum (tympanic membrane). The membrane was loaded with a P. aeruginosa suspension, which was allowed to grow for 4 days. The P. aeruginosa suspension was trapped in the well through the biofilm formation period.

The P. aeruginosa biofilm-loaded membrane closely resembles a bacterial-infected eardrum. The mean pore size of the membrane (8 μm) is larger than the lengths of the P. aeruginosa cells (— 3 μm length) inserted into the membrane. The consistent result of these tests was that no intact cells were observed after microplasma jet array treatment. Reactive species, such as OH and ¹O₂ (singlet oxygen) produced by the microplasma jet array, provide considerable oxidative pressure to the P. aeruginosa biofilm and result in the disruption of the biofilm Collapsed cell structures were observed after plasma array treatment.

The bottom side of the biofilm-loaded membrane was also examined before and after microplasma jet array treatment. Before treatment, the cellulose nitrate network and a permeated bacterial suspension coating was observed to be intact. However, following exposure of the bacteria to the plasma array, the cellulose structures were consistently observed to be at least partially dismantled (ruptured) which indicates that cell integrity had been lost.

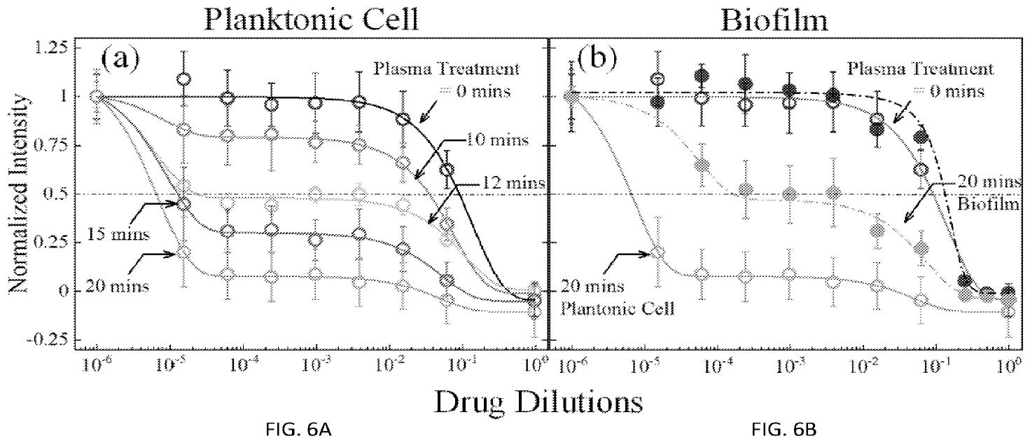

The drug-susceptibility of P. aeruginosa planktonic cells treated by a microplasma jet array for 0, 10, 12, 15, and 20 mins was measured and the results are plotted in FIG. 6A. The 50% minimal inhibitory concentrations (MIC₅₀) of the drug against the planktonic cells decreased from a dilution of 9×10⁻² to 3.4×10⁻² after 10 mins of microplasma jet array treatment. With longer treatment times of 12, 15, and 20 mins, the MIC₅₀ quickly dropped more than three orders of magnitudes to 2.28×10⁻⁵, 1.24×10⁻⁵, and 6.47×10⁻⁶ dilutions, respectively, as shown in FIG. 6A. These results demonstrate that 12-15 mins of microplasma jet array treatment can decrease the antibiotic dosage by more than 1000 times relative to that required for non-treated samples to achieve MIC₅₀.

FIG. 6B (solid circles) illustrates the drug-susceptibility of a 4-day-old P. aeruginosa biofilm that was either untreated or after 20 mins of microplasma jet array treatment. The drug-susceptibility response of the biofilm without treatment is illustrated by the black dashed line in FIG. 6B. Compared with the planktonic cells (FIG. 6A), the MIC₅₀ of the biofilm increased by a factor of four to a dilution of 1.33×10⁻¹, which is to be expected as the biofilm acts as a shelter for the planktonic cells that reside within it. Following exposure of the biofilm to 20 mins of microplasma jet array treatment, the MIC₅₀ decreased to 1.62×10⁻⁴ dilution, which is close to three orders-of-magnitude lower than the corresponding value of the biofilm without plasma treatment, as shown in FIG. 6B. Compared with isolated P. aeruginosa planktonic cells, the presence of the biofilm decreases the sensitivity to microplasma jet array treatment by about a factor of 25.

In summary, the introduction of low temperature plasma within the human ear canal and near the outer surface of the eardrum produces molecular radicals and molecules such as OH and NO, respectively. Tests conducted with membranes that act as a surrogate for the eardrum show that biofilms on the side of the membrane opposite from the plasma are at least partially disrupted and deactivated. Molecular species produced near the eardrum are able to diffuse through this thin membrane and at least partially destroy the bacterial cells responsible for ear infections. The effect of the plasma treatment on biofilms responsible for ear infections is to dramatically reduce the dependence on antibiotics. Consequently, the potential impact of this plasma otoscope in reducing treatment time and the role of antibiotics appears to be significant.

While specific embodiments of the present invention have been shown and described, it should be understood that other modifications, substitutions and alternatives are apparent to one of ordinary skill in the art. Such modifications, substitutions and alternatives can be made without departing from the spirit and scope of the invention, which should be determined from the appended claims.

Various features of the invention are set forth in the appended claims.

1. A speculum configured for attachment to an otoscope, the speculum comprising:
a speculum body configured to attach to the otoscope;
an array of radially situated microchannels within the speculum body and extending to apertures in a distal end of the speculum body;
a power electrode array within the speculum body positioned with respect to the microchannels to excite plasma generation within the microchannels; and
an optically transparent central portion in the body to permit viewing of an eardrum by a practitioner.

2. The speculum according to claim 1, wherein the material of the body isolates the power electrode array from the array of conically situated microchannels.

3. The speculum according to claim 1, comprising inlets to the microchannels to receive a gas flow and contacts to the power electrode array.

4. The speculum according to claim 3, operated on the otoscope to produce a micro jet output.

5. The speculum according to claim 1, wherein the optically transparent central portion comprises an empty lumen.

6. The speculum according to claim 1, wherein the power electrode array comprises an array of radially situated electrodes.

7. The speculum according to claim 6, wherein the radially situated electrodes and the radially situated microchannels alternate in an electrode section of the speculum.

8. The speculum according to claim 7, wherein the microchannels extend beyond the electrodes toward the distal end of the speculum body.

9. The speculum according to claim 7, wherein the microchannels extend beyond the electrodes toward a the distal end of the speculum body.

10. The speculum according to claim 1, comprising an adapter to connect to an otoscope, the adapter comprising:
a circumferential channel one side;
gas outlets on an opposite side, the gas outlets being in fluid communication with the circumferential gas channel and being sized any arranged to mate with the microchannels; and
positive engagement features to mate with the otoscope.

11. The speculum according to claim 10, wherein the otoscope is a conventional otoscope and the adapter is sized and configured to mate with the conventional otoscope.

12. The speculum according to claim 1, wherein the speculum is sized according to ear canal anatomy.

13. The speculum according to claim 12, wherein the speculum comprises a shape, material and tip to create a seal in the ear canal.

14. The speculum according to claim 12, wherein the electrode array extends a length away from the distal end that keeps the electrode array outside of the ear canal.

15. A method for biofilm treatment in the middle ear and middle ear cavity, the method comprising:
inspecting the middle ear of a subject through an otoscope;
judging whether treatment is necessary;
when treatment is necessary, actuating plasma jets to extend into the ear canal from a speculum attached to the otoscope;
continuing to plasma jet treatment for a period of time sufficient to inactivate or kill a bacterial biofilm in the middle ear and/or the middle ear cavity.

16. The method of claim 15, further comprising creating a seal in the ear canal with a tip of the speculum prior to the actuating of the plasma jets.

17. The method of claim 15, repeated on a single patient over a period of time.

18. The method of claim 15, wherein the speculum comprises:
a speculum body configured to attach to the otoscope;
an array of radially situated microchannels within the speculum body and extending to apertures in a distal end of the speculum body;
a power electrode array within the speculum body positioned with respect to the microchannels to excite plasma generation within the microchannels; and
an optically transparent central portion in the body to permit viewing of an eardrum by a practitioner
